(19) United States
(12) Patent Application Publication
(10) Pub. No.: US 2019/0274894 A1
(43) Pub. Date: Sep. 12, 2019

(54) SELF-SUPPORTING ABSORBENT ARTICLE SYSTEM

(71) Applicant: Kimberly-Clark Worldwide, Inc., Neenah, WI (US)

(72) Inventors: Inyoung Sa, SeongNam-si (KR); HyeJin Lee, Seoul-si (KR); CaiShan Tan, SeongNam-si (KR); HyoungKun Park, Yongin-si (KR); Nathaniel T. Hollingsworth, Neenah, WI (US); SeongDae Roh, Yongin (KR)

(21) Appl. No.: 16/344,575

(22) PCT Filed: Oct. 31, 2016
(86) PCT No.: PCT/US2016/059650

§ 371 Date: Apr. 24, 2019

(51) Int. Cl. A61F 13/47; A61F 13/472

(52) U.S. Cl. CPC A61F 13/4702; A61F 13/47254

(57) ABSTRACT

A self-supporting absorbent article system can have an absorbent article and a support frame. The support frame is capable of supporting the absorbent article in a position adjacent to the body of the wearer of the absorbent article without the need for either 1) the absorbent article to be placed into the wearer's undergarment for support or 2) utilization of any other waistband, straps, or adhesive to maintain the support frame in position on the wearer's body.

### BACKGROUND OF THE DISCLOSURE

Female underwear is generally intended to provide coverage for the female external genital organs and includes a variety of shapes and fits. Generally, female underwear includes a waistband, a front portion, a crotch portion, and a rear portion. Certain styles of underwear tend to show lines through tight, close-fitting clothing. For example, briefs, bikinis, and boyshorts style underwear tend to reveal a pantyline at the edge and/or edge seam of the underwear. Thong-style underwear (including a g-string, v-string, c-string, tanga, or T-back) do not include a front portion and do not cover the buttocks. In thong-style underwear, when worn properly by a wearer, the waistband encircles the waist of the wearer, the crotch portion extends downward from the front of the waistband and positions over the crotch of the wearer, the rear portion extends from the rear of the waistband to the posterior end of the crotch portion, where the crotch and rear portions attach. The rear portion of a thong-style underwear includes a string, thin band, or a thin strip of fabric that fits between the buttocks of the wearer. Also, thong-style underwear have an abbreviated crotch portion which has a substantially narrower posterior end compared to the anterior end. Thong-style underwear, thereby, reduce the appearance of pantylines.

Difficulty arises for the wearer of thong-style underwear when they begin their menstrual cycle. The wearer may desire to continue to wear thong-style underwear to avoid the appearance of pantylines, however, the thong-style underwear generally does not accommodate the usage of an absorbent article such as, for example, a feminine napkin or pantiliner. Generally, feminine napkins and pantiliners are provided with a garment attachment adhesive and are placed directly into the wearer's undergarment and held in place in the wearer's undergarment with the garment attachment adhesive. As the thong-style underwear does not cover the buttocks and has an abbreviated crotch portion with a substantially narrower posterior end, there is much less fabric available in the thong-style underwear to which to adhere an absorbent article. In situations in which the wearer is able to adhere an absorbent article to the thong-style underwear, the weight of the absorbent article can cause the string, the thin band, or the thin strip of fabric fitting between the buttocks to sag away from the wearer's body. Additionally, garment attachment adhesive located on the absorbent article may cause portions of the absorbent article to stick to itself rendering the absorbent article uncomfortable to wear and decreasing the ability of the absorbent article to perform as intended in capturing and absorbing body exudates.

There is a need to provide an absorbent article system to a wearer such that the wearer can continue to reduce the appearance of pantylines as well has protect their skin and clothing during her menstrual cycle.

### SUMMARY OF THE DISCLOSURE

In various embodiments, a self-supporting absorbent article system can have a support frame comprising a resilient frame material, an anterior portion, a posterior portion, and a crotch portion positioned between the anterior portion and the posterior portion, a length dimension and a width dimension wherein the anterior portion width is greater than the posterior portion width; an absorbent article comprising an anterior portion, a posterior portion, and a crotch portion positioned between the anterior portion and the posterior portion, a length dimension and a width dimension wherein the anterior portion width is greater than the posterior portion width, a topsheet layer, a backsheet layer, and an absorbent core positioned between the topsheet layer and the backsheet layer; and an attachment mechanism for bonding the absorbent article to the support frame wherein the attachment mechanism is at least one of an adhesive, a cohesive, a mechanical attachment, a tie-on mechanism, or a sleeve.

In various embodiments, a transverse direction end edge of the support frame extends beyond a transverse direction end edge of the absorbent article. In various embodiments, a portion of the support frame extending beyond a portion of the absorbent article contacts a wearer's skin directly and is free from adhesive. In various embodiments, a portion of the support frame extending beyond a portion of the absorbent article does not contain sharp edges.

In various embodiments, the posterior portion of the absorbent core comprises a hydrophobic material.

In various embodiments, the absorbent article is capable of being bonded to the support frame. In various embodiments, the absorbent article is capable of being detached from the support frame.

In various embodiments, the support frame comprises a material selected from a stainless steel or plastic. In various embodiments, a length of the support frame is less than about 400 mm. In various embodiments, a width of the support frame is variable and is less than about 80 mm. In various embodiments, a thickness of the support frame is less than about 5 mm. In various embodiments, a length of the absorbent article is from about 200 mm to about 350 mm. In various embodiments, a width of the absorbent article is from about 40 mm to about 150 mm.

### BRIEF DESCRIPTION OF DRAWINGS

FIG. 1 is a perspective view of an embodiment of a self-supporting absorbent article system when in a usage configuration.

FIG. 2 is a top down view of the self-supporting absorbent article system of FIG. 1 when the absorbent article system is in a flat configuration.

FIG. 3 is a bottom view of the self-supporting absorbent article system of FIG. 1 when the absorbent article system is in a flat configuration.

FIG. 4 is a bottom view of an embodiment of a self-supporting absorbent article system when the absorbent article system is in a flat configuration.

FIG. 5 is a bottom view of an embodiment of a self-supporting absorbent article system when the absorbent article system is in a flat configuration.

FIG. 6 is a bottom view of an embodiment of a self-supporting absorbent article system when the absorbent article system is in a flat configuration.

FIG. 7 is a bottom view of an embodiment of a self-supporting absorbent article system when the absorbent article system is in a flat configuration.

FIG. 8 is a bottom view of an embodiment of a self-supporting absorbent article system when the absorbent article system is in a flat configuration.

FIG. 9 is a bottom view of an embodiment of a self-supporting absorbent article system when the absorbent article system is in a flat configuration.

Repeat use of reference characters in the present specification and drawings is intended to represent the same or analogous features or elements of the disclosure.

### DETAILED DESCRIPTION OF THE DISCLOSURE

The present disclosure is directed towards a self-supporting absorbent article system. The self-supporting absorbent article system can have an absorbent article and a support frame. The support frame is capable of supporting the absorbent article in a position adjacent to the body of the wearer of the absorbent article without the need for either 1) the absorbent article to be placed into the wearer's undergarment for support or 2) utilization of any other waistband, straps, or adhesive to maintain the support frame in position on the wearer's body.

### Definitions

As used herein, the term “absorbent article” refers herein to a garment or other end-use personal care absorbent article, including, but not limited to, catamenial products, such as sanitary napkins, feminine pads, pantiliners, and panty shields, incontinence devices, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber. Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

### Self-Supporting Absorbent Article System:

The present disclosure is directed towards a self-supporting absorbent article system. The self-supporting absorbent article system can have an absorbent article and a support frame. The support frame is capable of supporting the absorbent article in a position adjacent to the body of the wearer of the absorbent article without the need for either 1) the absorbent article to be placed into the wearer's undergarment for support or 2) utilization of any other waistband, straps, or adhesive to maintain the support frame in position on the wearer's body.

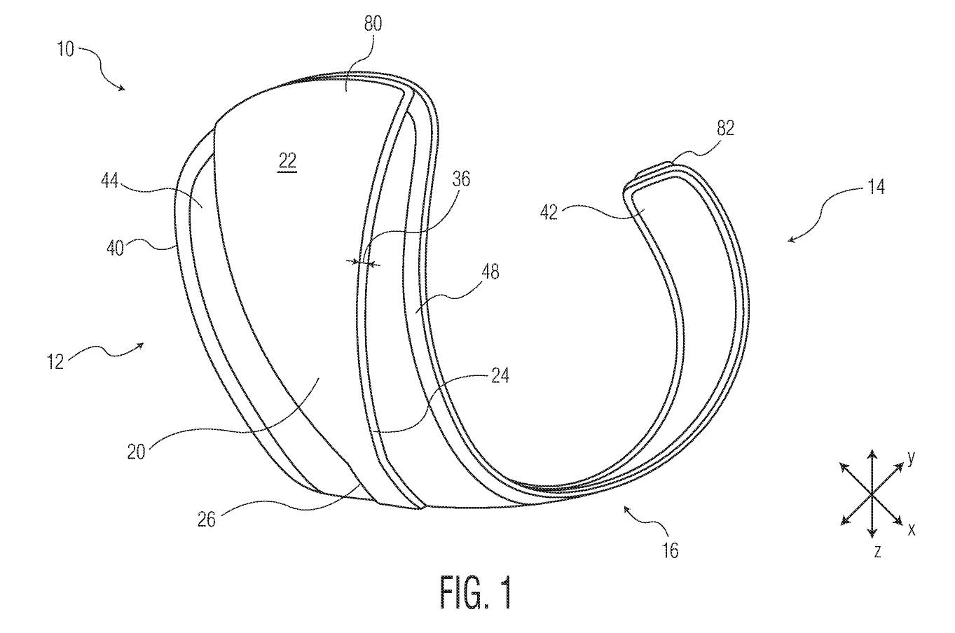

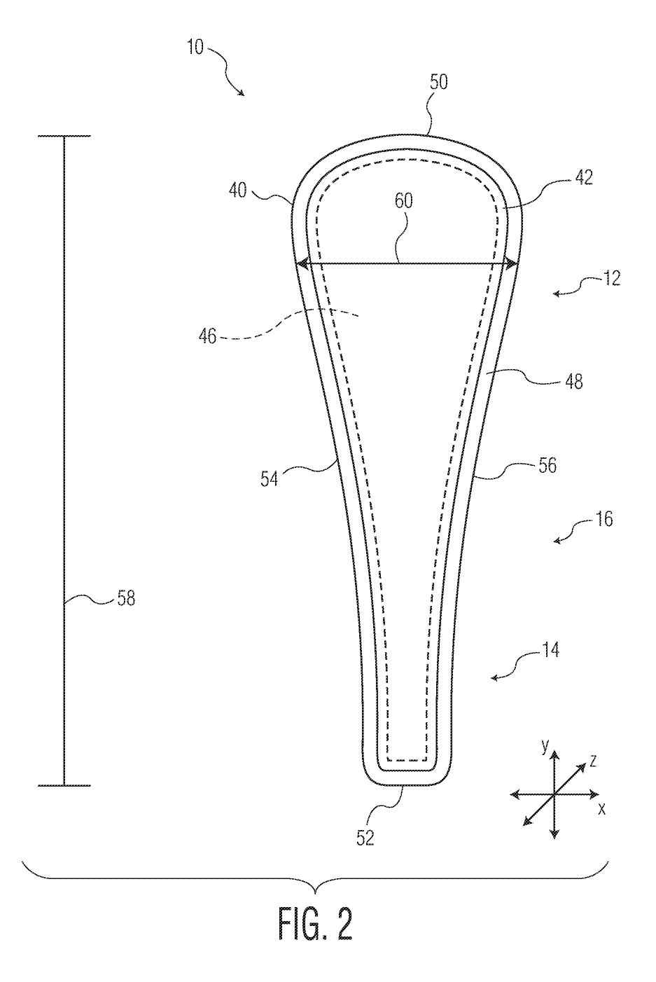

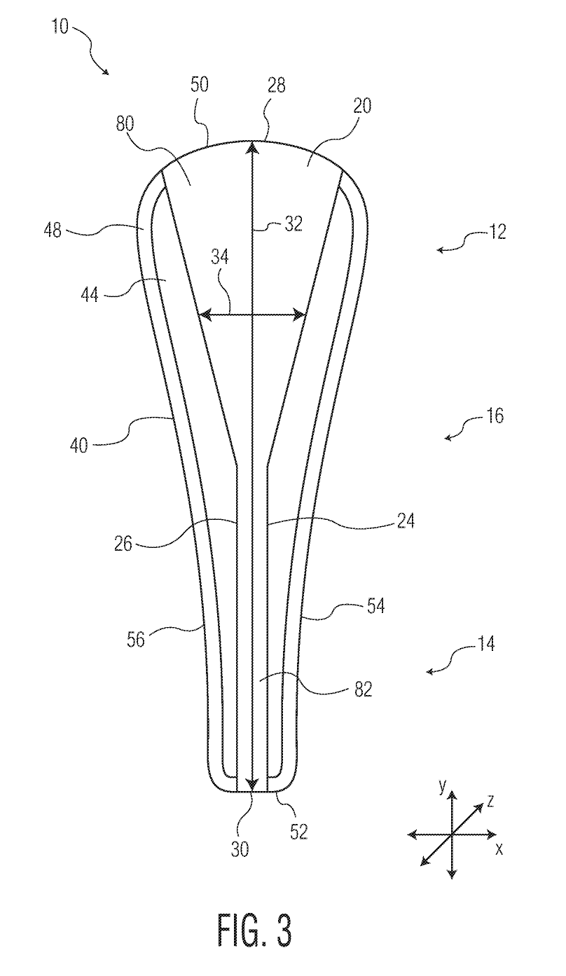

Referring to FIGS. 1-3, FIG. 1 provides an illustration of a perspective view of an exemplary self-supporting absorbent article system 10 in a usage configuration, FIG. 2 provides an illustration of a top down view of the self-supporting absorbent article system 10 of FIG. 1 in a flat configuration, and FIG. 3 provides an illustration of a bottom view of the self-supporting absorbent article system 10 of FIG. 1 in a flat configuration. The self-supporting absorbent article system 10 has an anterior portion 12, a posterior portion 14, and a crotch portion 16. When worn by a wearer, the crotch portion 16 will fit between the wearer's legs while the posterior portion 14 will curve upwardly along the cleavage of the buttocks of the wearer and the anterior portion 12 will curve upwardly along the front of the wearer's body. The self-supporting absorbent article system 10 can have a longitudinal direction (Y), a transverse direction (X), and a depth direction (Z).

The self-supporting absorbent article system 10 can have a support frame 20 and an absorbent article 40 which is supported by the support frame 20. The absorbent article 40 can be placed within the support frame 20 to form the self-supporting absorbent article system 10. For usage, the self-supporting absorbent article system 10 can then be placed directly onto the body of the wearer. The support frame 20 can maintain the absorbent article 40 adjacent to the body of the wearer without the need for utilizing any other waistband, straps, or adhesive to maintain such placement of the self-supporting absorbent article system 10 adjacent to the wearer's body and without the need for the for the wearer to don any additional undergarment to maintain placement of the self-supporting absorbent article system 10 adjacent to their body.

The support frame 20 can be formed from a material which will provide a resilient flexibility to the self-supporting absorbent article system 10 such as, for example, resilient stainless steel or a resilient plastic material. The support frame 20 needs to have the capability of conforming to the wearer's body as well as providing snug engagement between the self-supporting absorbent article system 10 and the wearer's body. The support frame 20 needs to be readily adaptable to the body of the wearer as well as permit relative flexure movement between the anterior portion 12 and the posterior portion 14 of the self-supporting absorbent article system 10. In usage, the self-supporting absorbent article system 10 is configured into a U-shape and is adapted to surround the genital region of the wearer. The anterior portion 12 and the posterior portion 14 can curve inwardly towards each other in a spring-biased arrangement. The anterior portion 12 and the posterior portion 14 of the self-supporting absorbent article system 10 need to be able to be move in a direction away from the wearer's body to allow for changing of the absorbent article 40 which is held by the support frame 20.

The support frame 20 can have a first transverse direction (X) end edge 28 in the anterior portion 12 and a second transverse direction (X) end edge 30 in the posterior portion 14, and a pair of opposing longitudinal direction side edges, 24 and 26, extending between the transverse direction end edges, 28 and 30. The support frame 20 can have a garment facing surface 22 and an absorbent article facing surface opposite the garment facing surface 22. The support frame 20 can have a length 32 measured from the first transverse direction end edge 28 to the second transverse direction end edge 30 and the length 32 can be less than about 400 mm. In order to provide a shape to the support frame 20 such that the self-supporting absorbent article system 10 can have a crotch portion 16 fitting between the thighs of the wearer and a posterior portion 14 fitting along the cleavage of the buttocks, the support frame 20 can be shaped as deemed suitable. The longitudinal direction side edges, 24 and 26, can be spaced apart from each other and can have a width 34 as measured from the first longitudinal direction side edge 24 to the second longitudinal direction side edge 26. In various embodiments, the width 34 of the support frame 20 can vary in the longitudinal direction (Y) between the first transverse direction end edge 28 and the second transverse direction end edge 30. In various embodiments, the width 34 of the support frame 20 between the first longitudinal direction side edge 24 and the second longitudinal direction side edge 26 can be from about 10 mm to about 80 mm. In various embodiment, the width 34 between the longitudinal direction side edges, 24 and 26, can vary between the anterior portion 12, posterior portion 14, and/or crotch portion 16. The longitudinal direction side edges, 24 and 26, can be further from each other in the anterior portion 12 in order to provide support for an absorbent article 40. The width 34 between the longitudinal direction side edges, 24 and 26, can narrow as the support frame 20 transitions from the anterior portion 12 to the crotch portion 14 in order for the self-supporting absorbent article system 10 to fit between the thighs of the wearer. The width 34 between the longitudinal direction side edges, 24 and 26, in the posterior portion 16 can either be narrower than or the same as the width 34 between the longitudinal direction side edges, 24 and 26, in the crotch portion 16 such as deemed suitable for the support frame 20 to fit along the cleavage of the buttocks. From the perspective of the crotch portion 16 of the self-supporting absorbent article system 10, in various embodiments, such as illustrated in FIGS. 1-3, the support frame 20 can have a first extension 80 which extends from the crotch portion 16 into the anterior portion 12 and can have a second extension 82 which extends from the crotch portion 16 into the posterior portion 14. In various embodiments, the first extension 80 of the support frame 20 can have a shape that is generally triangular. The widest portion of the triangle shape can be approximate to the first transverse direction end edge 28 and the narrowest portion of the triangle shape can be in the crotch portion 16 of the self-supporting absorbent article system 10. The triangular shape of the first extension 80 can provide stability to the anterior portion 12 of the self-supporting absorbent article system 10. As the first extension 80 of the support frame 20 can have a generally triangular shape, it can also have a variable width 34 in the longitudinal direction (Y). Also as described herein, the support frame 20 can have a second extension 82 which has a generally rectangular shape. The rectangular shape of the second extension 82 can allow for the self-supporting absorbent article system 10 to curve upwardly along the cleavage of the buttocks of the wearer. In various embodiments, the second extension 82, in a generally rectangular shape can have a consistent width 34 in the longitudinal direction (Y), however, the width 34 of the second extension 82 can vary from the width 34 of the first extension 80. In various embodiments, the width 34 of the anterior portion 12 of the support frame 20 is greater than the width 34 of the posterior portion 14 of the support frame 20. In various embodiments, the width 34 of the anterior portion 12 at the first transverse direction end edge 28 is greater than the width 34 of the posterior portion 12 at the second transverse direction end edge 30. The support frame 20 can have a thickness 36 in the depth direction (Z) as measured from the garment facing surface 22 to the absorbent article facing surface. In various embodiments, the thickness 36 can be from about 1 mm to about 5 mm.

An absorbent article 40 can be placed on the absorbent article facing surface of the support frame 20 and supported by the support frame 20 against the body of the wearer. The absorbent article 40 can have a first transverse direction end edge 50 in the anterior portion 12, a second transverse direction end edge 52 opposite the first transverse direction end edge 50 and in the posterior portion 14, and a pair of opposing longitudinal direction side edges, 54 and 56. The absorbent article 40 can have a wearer facing, liquid permeable topsheet layer 42 and a garment facing, liquid impermeable backsheet layer 44. An absorbent core 46 can be positioned between the topsheet layer 42 and the backsheet layer 44. The topsheet layer 42 and the backsheet layer 44 can both extend beyond the outermost peripheral edges of the absorbent core 46 and can be peripherally bonded together, either entirely or partially, using known bonding techniques to form a sealed peripheral region 48. For example, the topsheet layer 42 and the backsheet layer 44 can be bonded together by adhesive bonding, ultrasonic bonding, or any other suitable bonding method known in the art.

The topsheet layer 42 defines a wearer facing surface of the absorbent article 40 that may directly contact the body of the wearer and is liquid permeable to receive body exudates. The topsheet layer 42 is desirably provided for comfort and conformability and functions to direct body exudates away from the body of the wearer, through its own structure, and towards the absorbent core 46. The topsheet layer 42 desirably retains little to no liquid in its structure, so that it provides a relatively comfortable and non-irritating surface next to the skin of the wearer of the absorbent article 40.

The topsheet layer 42 can be a single layer of material, or alternatively, can be multiple layers that have been laminated together. The topsheet layer 42 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 42.

In various embodiments, the topsheet layer 42 can be constructed from various nonwoven webs such as meltblown webs, spunbond webs, hydroentangled spunlace webs, or through air bonded carded webs. Examples of suitable topsheet layer 42 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 42 can be a bonded carded web made of polypropylene and polyethylene such as that obtainable from Sandler Corporation, Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 42, each of which is hereby incorporated by reference thereto in its entirety. Additional topsheet layer 42 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 42 may contain a plurality of apertures (not shown) formed therethrough to permit body exudates to pass more readily into the absorbent core 46. The apertures may be randomly or uniformly arranged throughout the topsheet layer 42. The size, shape, diameter, and number of apertures may be varied to suit an absorbent article's 40 particular needs.

In various embodiments, the topsheet layer 42 can have a basis weight ranging from about 5, 10, 15, 20 or 25 gsm to about 50, 100, 120, 125 or 150 gsm. For example, in an embodiment, a topsheet layer 42 can be constructed from a through air bonded carded web having a basis weight ranging from about 15 gsm to about 100 gsm. In another example, a topsheet layer 42 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics and others.

In various embodiments, the topsheet layer 42 can be at least partially hydrophilic. In various embodiments, a portion of the topsheet layer 42 can be hydrophilic and a portion of the topsheet layer 42 can be hydrophobic. In various embodiments, the portions of the topsheet layer 42 which can be hydrophobic can be either an inherently hydrophobic material or can be a material treated with a hydrophobic coating.

An absorbent core 46 can be positioned between the topsheet layer 42 and the backsheet layer 44. The absorbent core 46 can generally be any single layer structure or combination of layer components, which can demonstrate some level of compressibility, conformability, be non-irritating to a wearer's skin, and capable of absorbing and retaining liquids and other body exudates. Additionally, the absorbent core 46 can provide additional capacity to absorb and retain body exudates such as menses. In various embodiments, the absorbent core 46 can be formed from a variety of different materials and can contain any number of desired layers. For example, the absorbent core 46 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers. In various embodiments, the posterior portion of the absorbent core 46 of the absorbent article 40 comprises a hydrophobic material.

In various embodiments, if desired, the absorbent core 46 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 46 in any amount as desired.

Regardless of the combination of absorbent materials used in the absorbent core 46, the absorbent materials can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

The shape of the absorbent core 46 can vary as desired and can comprise any one of various shapes including, but not limited to, triangular, rectangular, dog-bone and elliptical shapes. In various embodiments, the absorbent core 46 can have a shape that generally corresponds with the overall shape of the absorbent article 40. The dimensions of the absorbent core 46 can be substantially similar to those of the absorbent article 40, however, it will be appreciated that the dimensions of the absorbent core 46 while similar, will often be less than those of the overall absorbent article 40, in order to be adequately contained therein.

By way of example, suitable materials and/or structures for the absorbent core 46 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al., each of which is hereby incorporated by reference thereto in its entirety.

As described above, in various embodiments, an absorbent core 46 can be a single layer structure and can include, for example, a matrix of cellulosic fluff and superabsorbent material. In various embodiments, an absorbent core 46 can have at least two layers of material, such as, for example, a body facing layer and a garment facing layer. In various embodiments, the two layers can be identical to each other. In various embodiments, the two layers can be different from each other. In such embodiments, the two layers can provide the absorbent article 40 with different absorption properties as deemed suitable. In various embodiments, the body facing layer of the absorbent core 46 may be constructed of an airlaid material and the garment facing layer of the absorbent core 34 may be constructed of a superabsorbent polymer-containing compressed sheet. In such embodiments, the airlaid material can have a basis weight from about 40 to about 200 gsm and the superabsorbent polymer-containing compressed sheet can be a cellulosic fluff based material that can be a combination of cellulosic pulp and SAP enclosed with a tissue carrier and having a basis weight from about 40 to about 400 gsm.

The backsheet layer 44 is generally liquid impermeable and is the portion of the absorbent article 40 which faces the support frame 20 and garment of the wearer. The backsheet layer 44 can permit the passage of air or vapor out of the absorbent article 40 while still blocking the passage of liquids. Any liquid impermeable material may generally be utilized to form the backsheet layer 44. The backsheet layer 44 can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the backsheet layer 44 can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a backsheet layer 44 can be a polyethylene film such as that obtainable from Pliant Corporation, Schaumburg, Ill., USA. Another example can include calcium carbonate-filled polypropylene film. In still another embodiment, the backsheet layer 44 can be a hydrophobic nonwoven material with water barrier properties such as a nonwoven laminate, an example of which can be a spunbond, meltblown, meltblown, spunbond, four-layered laminate. The backsheet layer 44 can, therefore, be of a single or multiple layer construction, such as of multiple film layers or laminates of film and nonwoven fibrous layers. Suitable backsheet layers 44 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

In order to provide a shape to the absorbent article 40 such that the self-supporting absorbent article system 10 can have a crotch portion 16 fitting between the thighs of the wearer and a posterior portion 14 fitting along the cleavage of the buttocks, the absorbent article 40 can be shaped as deemed suitable. The absorbent article 40 can have a length 58 measured from the first transverse direction end edge 50 to the second transverse direction end edge 52 and the length 58 can be from about 200 to about 400 mm. The longitudinal direction side edges, 54 and 56, can be spaced apart from each other and can have a width 60 measured from the first longitudinal directions ide edge 54 to the second longitudinal direction side edge 56. In various embodiments, the width 60 of the absorbent article 40 can vary in the longitudinal direction (Y) between the first transverse direction end edge 50 and the second transverse direction end edge 52. In various embodiments, the width 60 of the absorbent article 40 can be from about 40 mm to about 150 mm. In various embodiments, the width 60 between the longitudinal direction side edges, 54 and 56, can vary between the anterior portion 12, posterior portion 14, and/or crotch portion 16. The longitudinal direction side edges, 54 and 56, can be further from each other in the anterior portion 12. The width 60 between the longitudinal direction side edges, 54 and 56, can narrow as the absorbent article 40 transitions from the anterior portion 12 to the crotch portion 14 in order for the self-supporting absorbent article system 10 to fit between the thighs of the wearer. The width 60 between the longitudinal direction side edges, 54 and 56, in the posterior portion 16 can either be narrower than or the same as the width 60 between the longitudinal direction side edges, 54 and 56, in the crotch portion 16 such as deemed suitable for the absorbent article 40 to fit along the cleavage of the buttocks.

From the perspective of the crotch portion 16 of the self-supporting absorbent article system 10, in various embodiments such as illustrated in FIGS. 1-3, the absorbent article 40 can have a generally triangular shape with a larger portion in the anterior portion 12 than in the posterior portion 14. The widest portion of the triangle shape can be approximate to the first transverse direction end edge 50 and the narrowest portion of the triangle shape can be approximate to the second transverse direction end edge 52.

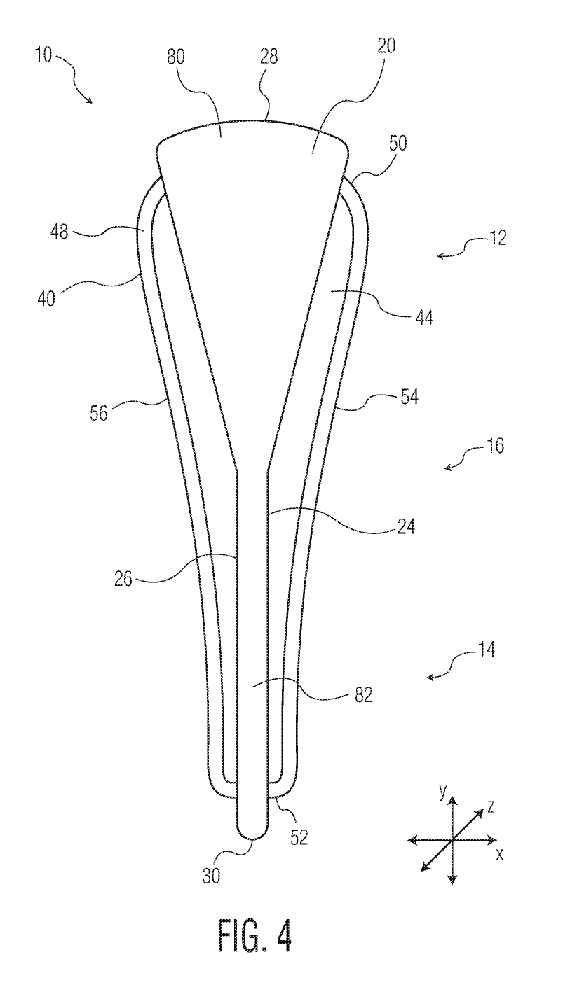

Referring to FIG. 4, in various embodiments, the transverse direction end edges, 28 and 30, of the support frame 20 can extend beyond the transverse direction end edges, 50 and 52, of the absorbent article 40. In such embodiments, the support frame 40 can come into direct contact with the skin of the wearer of the self-supporting absorbent article system 10. As the self-supporting absorbent article system 10 can remain on the body of the wearer without the need of a waistband, straps, or adhesive, the portions of the support frame 20 extending beyond the absorbent article 40 can be free of adhesive.

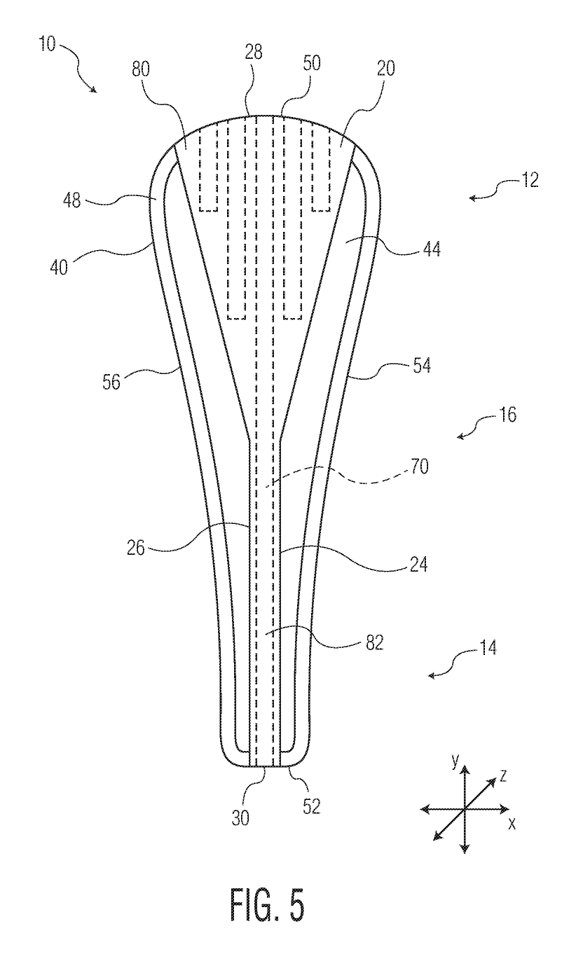

Referring to FIG. 5, in various embodiments, the absorbent article 40 is bonded to the support frame 20 via an attachment mechanism 70. In various embodiments, the attachment mechanism 70 can be at least one of an adhesive, cohesive, mechanical fastener such as, for example, a hook-and-loop cooperative system, and/or a tie-on mechanism. In various embodiments, the attachment mechanism 70 such as, for example, an adhesive, can be positioned on solely the backsheet layer 44 of the absorbent article 40. In various embodiments, the attachment mechanism 70 such as, for example, an adhesive, can be positioned solely on the absorbent article facing surface of the support frame 20. In various embodiments, the attachment mechanism 70 such as, for example, a cohesive can be positioned on each of the backsheet layer 44 and the absorbent article facing surface of the support frame 20.

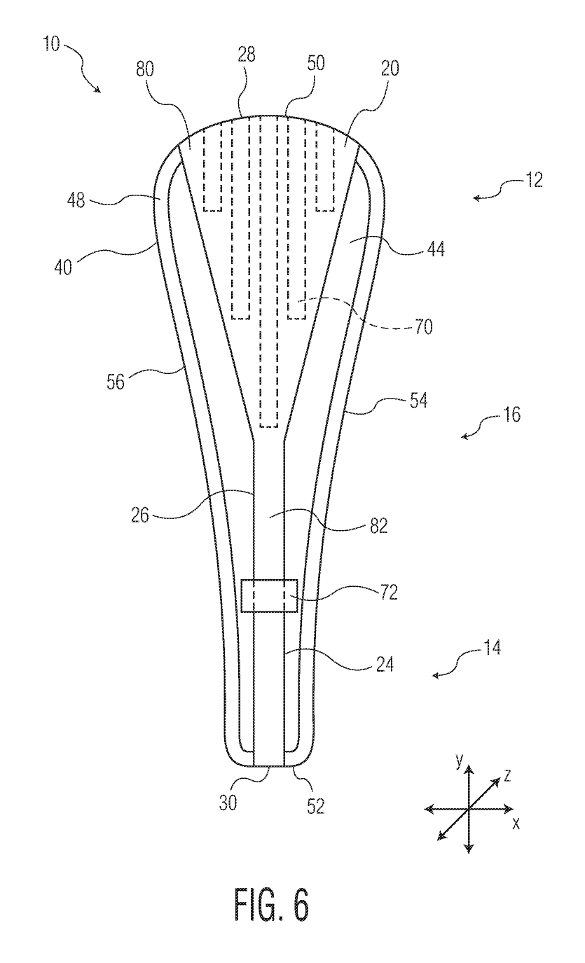

Referring to FIG. 6, in various embodiments, the absorbent article 40 is bonded to the support frame 20 via an attachment mechanism 70 which is positioned on the backsheet layer 44 of the absorbent article 40 and/or on the absorbent article facing surface of the support frame 20. Additionally, the backsheet layer 44 may be provided with a sleeve 72 through which the second extension 82 of the support frame 20 may extend. The sleeve 72 may formed by bonding a piece of material, such as described herein for the topsheet layer 42 or the backsheet layer 44, to the backsheet layer 44 of the absorbent article 40. The longitudinal direction (Y) edges of the sleeve 72 may be bonded to the backsheet layer 44 of the absorbent article 40, however, the transverse direction (X) edges of the sleeve 72 may remain free from bonding such that the second extension 82 of the support frame 20 may pass between the material of the sleeve 72 and the backsheet layer 44 during assembly by the wearer of the self-supporting absorbent article system 10. In such embodiments, the posterior portion 14 of the support frame 20 is held in place via the usage of the sleeve 72. The sleeve 72 can be any dimension as deemed suitable. In various embodiments, the sleeve 72 can have a width dimension in the transverse direction (X) which can be just wider than the width 34 of the second extension 82. Such a dimensioning of the sleeve 72 can maintain a close relationship between the support frame 20 and the absorbent article 40 and can prevent movement of the second section 82 of the support frame 20. In various embodiments, the sleeve 72 can have a width dimension in the transverse direction (X) which can be much wider than the width 34 of the second extension 82 of the support frame 20. Such a dimensioning of the sleeve 72 can allow for movement of the support frame 20 while the self-supporting absorbent article system 10 is in use. In various embodiments, such movement of the support frame 20 may be desirable as it may promote movement of the absorbent article 40 such that it forms a bridge and moves closer to the body of the wearer.

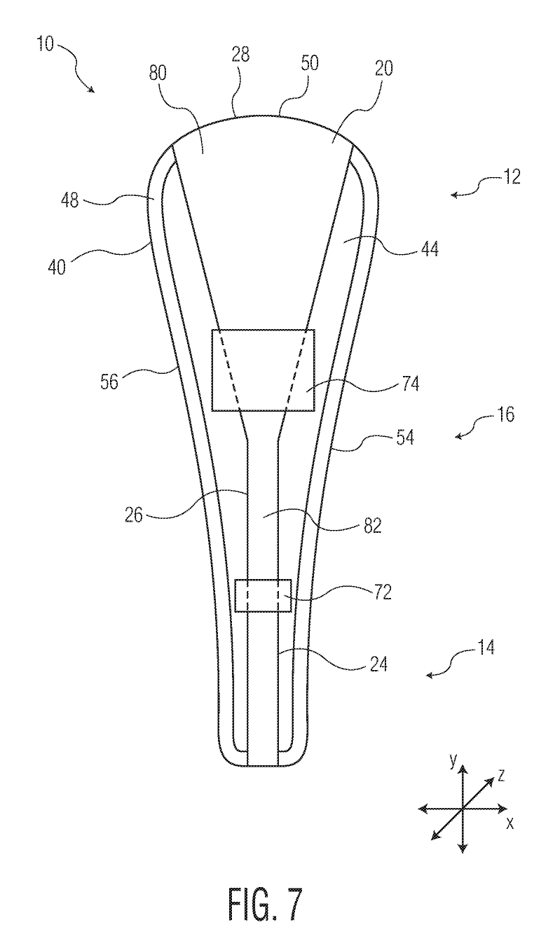

Referring to FIG. 7, in various embodiments, the absorbent article 40 is bonded to the support frame 20 via the usage of multiple sleeves, 72 and 74, bonded to the backsheet layer 44 of the absorbent article 40. The sleeves, 72 and 74, can be positioned in any location of the backsheet layer 44 as deemed suitable to maintain the support frame 20 in the proper location. For example, a sleeve 72 can be positioned in the posterior portion 14 of the absorbent article 40 and another sleeve 74 can be positioned in the crotch portion 16 of the absorbent article 40. In various embodiments, at least 1, 2, 3, 4, or 5 sleeves, 72 and 74, can be bonded to the backsheet layer 44 of the absorbent article 40. In various embodiments, the sleeves, 72 and 74, can be provided in any shape as deemed suitable. As a non-limiting example, referring to FIG. 7, sleeve 72 is illustrated in the shape of a rectangle and sleeve 74 is illustrated in the shape of a square. The sleeves, 72 and 74, can be any suitable shape, such as, but not limited to, square, rectangle, trapezoid, etc. In various embodiments, the sleeve, 72 and 74, can be a shape which closely approximates the shape of the support frame 20 such that the wearer of the self-supporting absorbent article system 10 can obtain correct placement and positioning of the support frame 20 within the sleeve, 72 and 74, during usage.

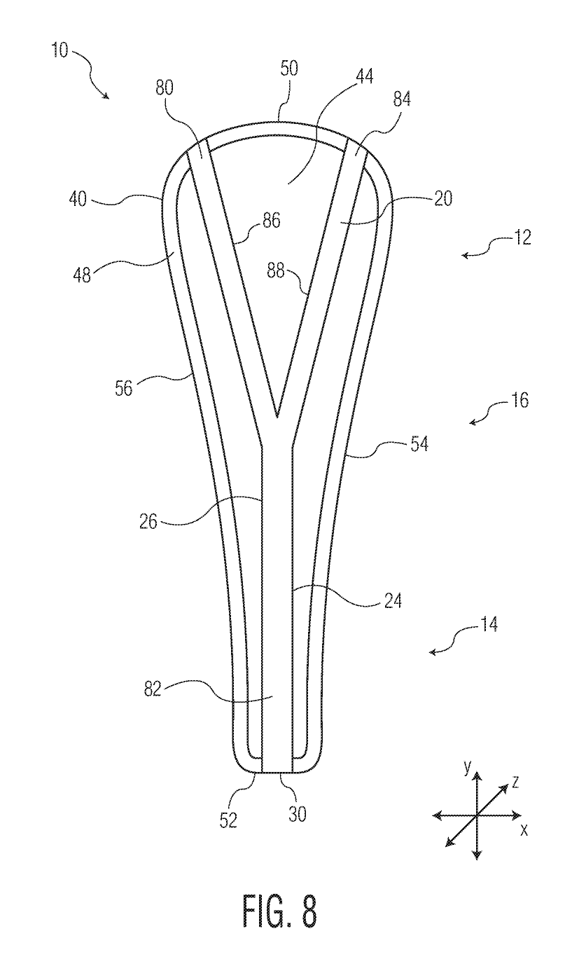

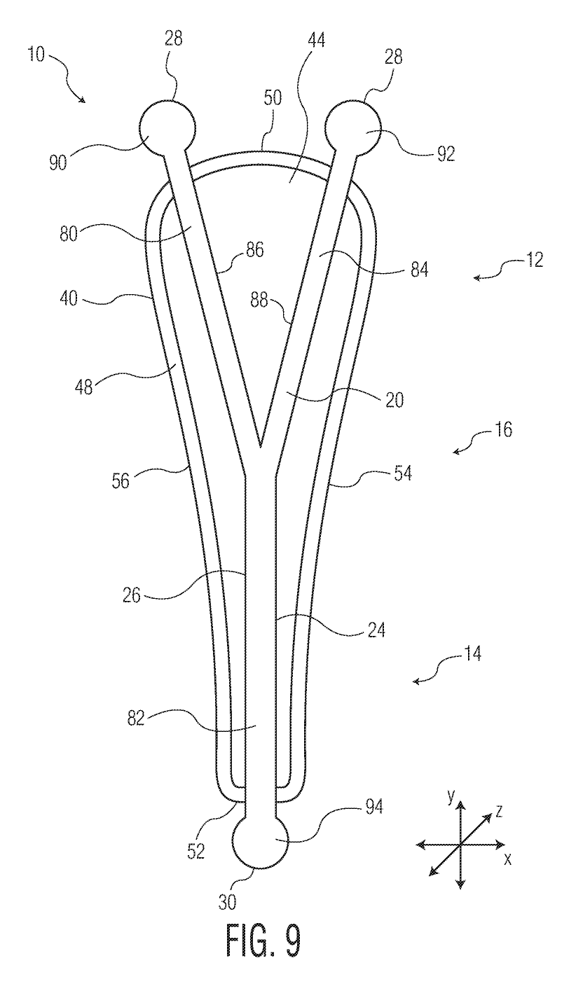

Referring to FIGS. 1 and 3-7, the anterior portion 12 of the support frame 20 can be in the general shape of a triangle. Referring to FIGS. 8 and 9, in various embodiments, the anterior portion 12 of the support frame 20 can be in the generally shape of a “V”. In such embodiments, from the perspective of the crotch portion 16 of the support frame 20, the support frame 20 has three extensions—a first extension 80 extending into the anterior portion 12, a second extension 82 extending into the posterior portion 14, and a third extension 84 also extending into the anterior portion 12 and spaced apart from the first extension 80. Longitudinally extending sidewalls, 86 and 88, can further define the interior perimeter of each of the first extension 80 and the third extension 84. In various embodiments, the extensions, 80, 82, and 84, of the support frame 20 can extend beyond the transverse edges, 50 and 52, of the absorbent article 40. The portions of the support frame 20 extending beyond the end edges, 50 and 52, of the absorbent article 40 can be projections 90, 92, and 94, which can be rounded so as to minimize any negative effect resulting from contact with the wearer's skin.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. A self-supporting absorbent article system comprising:
a) a support frame comprising a resilient frame material, an anterior portion, a posterior portion, and a crotch portion positioned between the anterior portion and the posterior portion, a length dimension and a width dimension wherein the anterior portion width is greater than the posterior portion width;
b) an absorbent article comprising an anterior portion, a posterior portion, and a crotch portion positioned between the anterior portion and the posterior portion, a length dimension and a width dimension wherein the anterior portion width is greater than the posterior portion width, a topsheet layer, a backsheet layer, and an absorbent core positioned between the topsheet layer and the backsheet layer; and
c) an attachment mechanism for bonding the absorbent article to the support frame wherein the attachment mechanism is at least one of an adhesive, a cohesive, a mechanical attachment, a tie-on mechanism, or a sleeve.

2. The self-supporting absorbent article system of claim 1 wherein a transverse direction end edge of the support frame extends beyond a transverse direction end edge of the absorbent article.

3. The self-supporting absorbent article system of claim 2 wherein a portion of the support frame extending beyond a portion of the absorbent article contacts a wearer's skin directly and is free from adhesive.

4. The self-supporting absorbent article system of claim 2 wherein a portion of the support frame extending beyond a portion of the absorbent article does not contain sharp edges.

5. The self-supporting absorbent article system of claim 1 wherein the posterior portion of the absorbent core comprises a hydrophobic material.

6. The self-supporting absorbent article system of claim 1 wherein the absorbent article is capable of being bonded to the support frame.

7. The self-supporting absorbent article system of claim 6 wherein the absorbent article is capable of being detached from the support frame.

8. The self-supporting absorbent article system of claim 1 wherein the support frame comprises a material selected from a stainless steel or plastic.

9. The self-supporting absorbent article system of claim 1 wherein a length of the support frame is less than about 400 mm.

10. The self-supporting absorbent article system of claim 1 wherein a width of the support frame is variable and is less than about 80 mm.

11. The self-supporting absorbent article system of claim 1 wherein a thickness of the support frame is less than about 5 mm.

12. The self-supporting absorbent article system of claim 1 wherein a length of the absorbent article is from about 200 mm to about 350 mm.

13. The self-supporting absorbent article system of claim 1 wherein a width of the absorbent article is from about 40 mm to about 150 mm.
